(19) United States
(12) Patent Application Publication
(10) Pub. No.: US 2026/0027538 A1
(43) Pub. Date: Jan. 29, 2026

(54) FIXED BED REACTOR BASED ON THE PRINCIPLE OF THERMOLUMINESCENCE FOR IN-SITU HEAT REMOVAL AND IN-SITU TEMPERATURE MEASUREMENT OF STRONG EXOTHERMIC REACTIONS

(71) Applicants: Quzhou Institute for Innovation in Resource Chemical Engineering, Quzhou (CN); Beijing University of Chemical Technology, Beijing (CN)

(72) Inventors: Junting FENG, Beijing (CN); Qian WANG, Beijing (CN); Lulu XIE, Beijing (CN); Fengyu ZHANG, Beijing (CN); Xiaomeng CHEN, Beijing (CN); Xue DUAN, Beijing (CN)

(73) Assignees: Quzhou Institute for Innovation in Resource Chemical Engineering, Quzhou (CN); Beijing University of Chemical Technology, Beijing (CN)

(21) Appl. No.: 19/277,820

(22) Filed: Jul. 23, 2025

(30) Foreign Application Priority Data
Jul. 29, 2024 (CN) 202411019844.0

(51) Int. Cl. B01J 8/06; B01J 8/00; C07C 5/09; G01K 11/20

(52) U.S. Cl. CPC B01J 8/067; B01J 8/001; B01J 8/065; C07C 5/09; G01K 11/20; B01J 2208/00044; B01J 2208/00212; B01J 2208/00513; B01J 2208/065

(57) ABSTRACT

A fixed bed reactor based on the principle of thermoluminescence to achieve in-situ heat removal and in-situ temperature measurement for strong exothermic reactions can monitor the temperature and heat of the exothermic reaction process from multiple angles, enhance the heat transfer of the catalytic bed layer, and effectively reduce or eliminate the hot spots generated by the exothermic reaction process. This reactor includes a fixed bed reaction tube, and the top of the fixed bed reaction tube is equipped with a xenon lamp pretreatment system, an optical signal detection system, and an infrared temperature measurement system.

### CROSS-REFERENCE TO RELATED APPLICATIONS

This application claims priority to Chinese Patent Application No. 202411019844.0, filed on Jul. 29, 2024, the content of which is incorporated herein by reference in its entirety.

### TECHNICAL FIELD

The present application belongs to a method suitable for achieving gas phase exothermic reaction with in-situ heat removal and in-situ temperature measurement through thermoluminescence. Specifically, it relates to a fixed bed reactor based on the principle of thermoluminescence for in-situ heat removal and in-situ temperature measurement of strong exothermic reactions.

### BACKGROUND

Many chemical processes are strongly exothermic reactions, such as acetylene hydrogenation. For strongly exothermic reactions, as the reaction progresses, a large amount of heat is released. If not removed in time, the temperature of the catalyst bed will rise, leading to the generation of “hot spots”. High temperature difference not only increases a large amount of additional energy consumption, but also brings significant temperature changes and heat shock, which can easily cause catalyst failure, unstable reaction conversion rate and selectivity, and reduced system efficiency. More seriously, the high temperature and pressure changes during the reaction process can easily cause system overheating, pressure loss, and other problems, ranging from minor damage to process equipment to major safety accidents such as explosions, fires, and casualties. Therefore, in order to ensure the normal progress of the catalytic reaction process and improve reaction efficiency, it is necessary to remove the reaction heat in a timely manner.

For strongly exothermic reactions, many types of reactors have been designed and many methods for heat removal have been proposed. Chinese Patent Application Publication CN1736574A and CN207756118U propose a tubular fixed bed reactor, which forms a catalyst bed layer by filling the gap between the sleeves with catalyst. The cooling medium flows outside the tubes for heat transfer, which can increase the heat transfer area of the fixed bed reactor, shorten the heat transfer path, and make the reaction temperature axis radially uniform. However, this will bring difficulties in processing and greatly affect the effective loading amount of catalyst. Chinese Patent Application Publication CN2415831 proposes adding superconducting heat fins to the reactor cylinder, which can dissipate the heat released by the reaction outside the reactor. The heat dissipation capacity is more than 5 times that of a tubular reactor, but the heat transfer fins only remove some of the surrounding heat and cannot solve the “hot spots” in the catalyst bed. Patent Chinese Patent Application Publication CN1260237A proposes using thermally conductive metal particles as inert diluents in exothermic reactions to reduce and avoid the formation of hot spots in a fixed bed. This method only transfers heat from the catalytic active sites to the diluent and does not fully utilize the heat. Thermoluminescence refers to the release of electromagnetic radiation or other ionizing radiation that was originally absorbed and stored in lattice defects when a crystal is heated, in the form of photons. Based on thermoluminescence, heat removal is achieved through exothermic reactions, allowing for the reuse of heat. At the same time, in recent decades, temperature measurement through luminescence has gradually become an important part of the temperature measurement field. This temperature measurement method has high spatial resolution and temperature sensitivity compared to traditional thermocouple temperature measurement methods. In addition, a strip temperature measurement method based on photoluminescence is also proposed, but this luminescence process requires continuous excitation from the excitation source, which inevitably generates additional heat. By using thermoluminescence to remove heat from exothermic reactions and simultaneously measure temperature, additional heat generation issues can be avoided.

### SUMMARY

The purpose of the present application is to provide a fixed bed temperature measuring reactor based on the concept of thermoluminescence, which can enhance the heat transfer of the catalytic bed layer, physically mix the thermoluminescent material with the catalyst, convert thermal energy into light energy through thermoluminescence, and achieve the removal of heat from exothermic reactions. On the other hand, temperature measurement can be achieved by in-situ monitoring of the reaction process through changes in the light intensity of thermally luminescent materials.

To achieve the above objectives, the present application provides the following technical solution.

A fixed bed reactor suitable for in-situ heat removal and in-situ temperature measurement in gas-phase exothermic reactions includes a heating jacket and a fixed bed reaction tube. A heating jacket is installed outside the fixed bed reaction tube. A catalyst bed is installed inside the fixed bed reaction tube. A catalyst and a thermally luminescent material are placed inside the catalyst bed layer. The two ends of the fixed bed reaction tube are respectively equipped with inlet and outlet ports, with inlet at the side and outlet at the lower end. A thermocouple is installed inside the fixed bed reaction tube, with one end extending to the catalyst bed and the other end extending outside the fixed bed reaction tube. The fixed bed reactor further comprises at least one of a xenon lamp pretreatment system, an optical signal detection system, and an infrared temperature measurement system.

The xenon lamp pretreatment system includes a xenon lamp. When pretreatment of thermally luminescent materials is required, align the xenon lamp probe with the catalyst bed inside the reaction tube. After irradiating the stored electrons, the catalytic bed is then heated by introducing an inert gas or reaction gas atmosphere.

The optical signal detection system includes a reflector, a condenser, a monochromator, and a detector. When the top of the fixed bed reaction tube is made of quartz, measure the light intensity. The optical signal collection system is located at the top of the reaction tube, with the reflector and condenser in the darkroom. The thermochromic material in the catalytic bed emits light due to the release of electrons after heating up, which is refracted 90° by the reflector and converged by the condenser into the monochromator. The measured wavelength of light is selected and the intensity of the optical signal is measured by a detector. The detector converts the optical signal into an electrical signal to achieve optical signal intensity detection.

The constant temperature of the catalytic bed is a basic requirement for ensuring the normal operation of the catalytic process, and the lens of the infrared thermometer 13 is relatively large. If infrared temperature measurement is carried out on the side of the fixed bed, it will affect the insulation effect of the heating furnace, thereby affecting the progress of the catalytic process. As mentioned earlier, the present invention measures temperature and optical signals at the top of the reaction tube in a fixed bed.

The catalysts for acetylene hydrogenation to ethylene are Pd based, Ni based, Cu based catalysts, and the thermally luminescent materials are inorganic oxides doped with rare earth elements such as calcium fluoride, lithium fluoride, calcium sulfate, lithium borate, etc. The catalyst loading amount is 0.1 g-0.5 g, and the loading amount of the luminescent material is 0.1 g-1 g. The catalyst is physically mixed with the luminescent material or rare earth elements are doped or atoms are deposited on the catalyst to remove heat and measure the temperature of the exothermic reaction.

Compared with existing technologies, based on the principle of thermoluminescence, this application proposes to remove the heat of exothermic reactions by mixing thermoluminescent materials with catalysts, while ensuring the normal progress of catalytic reactions and not increasing the processing difficulty of the reactor, thus avoiding the generation of hot spots in the catalytic bed.

The present application combines infrared temperature measurement, optical signal detection, and fixed bed reactor to design the inner diameter, material, and inlet and outlet modules of the fixed bed reaction tube to meet the detection conditions, facilitate real-time monitoring of the reaction process, measure temperature from multiple angles, and obtain the true temperature of the catalytic reaction, further analyze the catalytic process, and improve the objective effectiveness of catalyst performance evaluation.

To ensure that the material of the reaction tube meets both light signal and temperature detection requirements, the top of the fixed bed reaction tube is equipped with a replaceable window. When replacing with germanium glass window panels, perform infrared temperature measurement to ensure the normal operation of the infrared temperature measurement process. When replaced with quartz window panels for light intensity detection, light is introduced into a monochromator and photomultiplier tube through a reflective mirror. The light signal is converted into an electrical signal for light intensity measurement. The actual temperature of the reaction process is obtained by comparing the light intensity during the reaction process with the light intensity at different temperatures in an inert atmosphere.

### BRIEF DESCRIPTION OF THE DRAWINGS

FIG. 1 is a schematic diagram of the fixed bed reactor and xenon lamp pretreatment system of the present application.

FIG. 2 is a schematic diagram of the fixed bed reactor and optical signal measurement system of the present application.

FIG. 3 is a schematic diagram of the fixed bed reactor and infrared temperature measurement system of the present application.

FIG. 4 shows the photocurrent testing of different materials.

FIG. 5 shows the photoluminescence spectra of different materials.

FIG. 6 shows the thermal release spectrum of LiF.

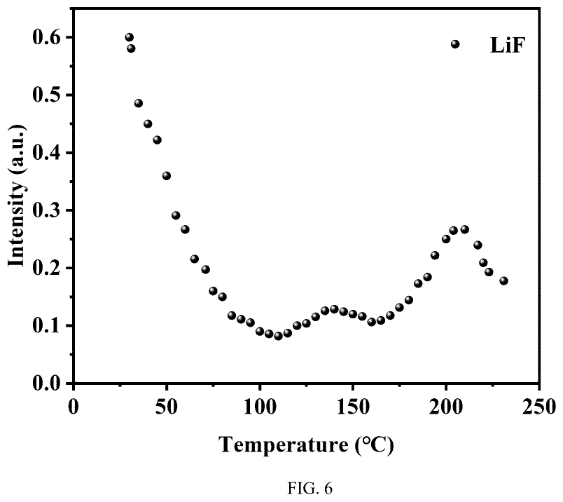

FIG. 7 shows the thermal release spectrum of CaF2.

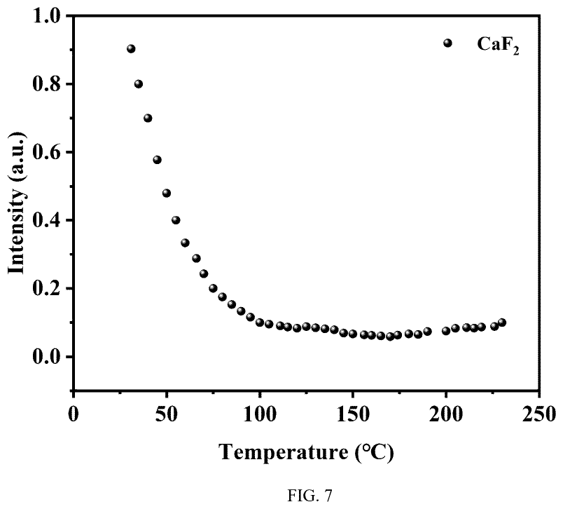

### DETAILED DESCRIPTION OF THE EMBODIMENTS

The following provides a further detailed explanation of the novel technical solution of the present application, but it is not intended to be a limitation of the present application.

A fixed bed reactor suitable for achieving strong exothermic reaction in situ heat removal through thermoluminescence includes a fixed bed reaction tube, and the top of the fixed bed reaction tube is equipped with one of a xenon lamp pretreatment system, a light signal detection system, and an infrared temperature measurement system. The side end of the fixed bed reaction tube is the inlet, and the lower end is the outlet. The fixed bed reaction tube is equipped with a thermocouple inlet. A catalyst bed 3 is installed inside the reaction tube, which is filled with catalyst. A heating jacket 4 is installed outside the fixed bed reaction tube 2, and a thermocouple is installed below the fixed bed reaction tube. The optical signal detection system sequentially includes a reflector 8, a spotlight 9, a monochromator 10, and a detector 11. Mirror 8 and spotlight 9 are set in darkroom 12.

The optical signal or temperature is measured at the top of the fixed bed reaction tube, and there is an inlet on the side of the fixed bed reaction tube to ensure the temperature of the catalytic bed layer.

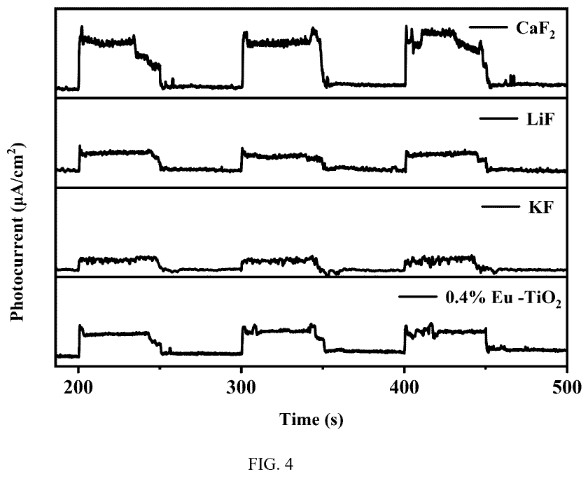

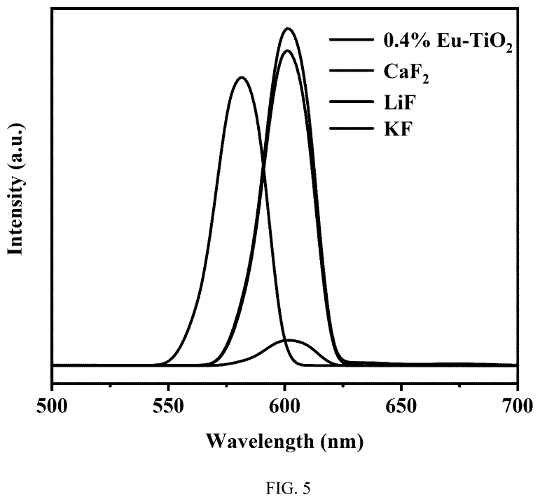

The ability of the thermally luminescent material to store electrons was determined through photocurrent testing and photoluminescence spectroscopy, as shown in FIGS. 4 and 5.

### Example 1

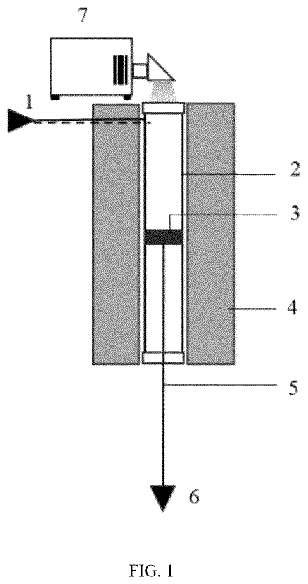

As shown in FIG. 1, it is a xenon lamp pretreatment system. The xenon lamp pretreatment system includes xenon lamp 7. When pre-treatment of thermally luminescent materials is required, align the xenon lamp probe with the catalyst bed inside the reaction tube to ensure uniform irradiation. According to the trap energy level depth formula

$$E = \frac{2 k {T}_{n}^{2}}{δ} ,$$

where k is the Boltzmann constant, Tn is the temperature value corresponding to the peak of the thermoluminescence curve, and δ is a parameter related to the temperature at half the peak intensity. The depth of defects in different materials varies, and the amount of electrons they can store also differs. Based on the principle of thermoluminescence, luminescent materials need to store electrons and release them to recombine with holes during the heating process to emit light. luminescent materials are illuminated by ultraviolet rays to store electrons. The amount of electrons stored in luminescent materials can be observed based on the height of the peak of the thermoluminescence curve observed at different irradiation times. When the peak intensity is the highest, it represents the highest amount of electron storage at this time, and the optimal time for ultraviolet treatment is at this time. After storing electrons under ultraviolet irradiation, inert gas or reaction gas atmosphere is then introduced to heat up the catalytic bed.

### Example 2

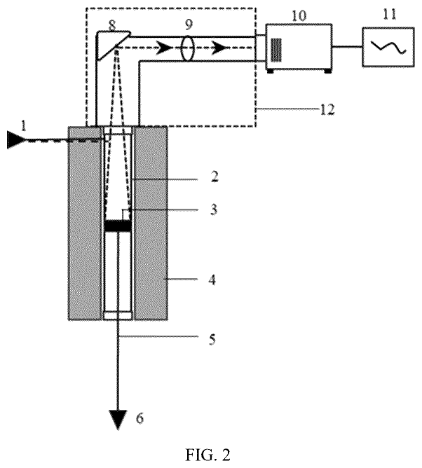

As shown in FIG. 2, it is an optical signal detection system. The optical signal measurement system includes a reflector 8, a condenser 9, a monochromator 10, a detector 11, and a darkroom 12. As mentioned earlier, the top of the fixed bed reaction tube is made of replaceable material, and when replaced with quartz, light intensity measurement is performed. The optical signal collection system is located at the top of reaction tube 2, and the entire optical signal collection part is in darkroom 12. The thermochromic material in catalytic bed 3 emits light due to the release of electrons after heating up, which is refracted by mirror 8 at a 90° angle and converged by condenser 9 before being introduced into monochromator 10. The measured wavelength of light is selected and the optical signal intensity is measured by detector 11. The detector converts the optical signal into an electrical signal to detect the intensity of the optical signal. Based on the principle of exothermic reaction and thermoluminescence, the heat released by the exothermic reaction is removed by converting thermal energy into light energy. At the same time, temperature measurement can be achieved by comparing the intensity of light signal detection under inert atmosphere and reactive gas atmosphere.

### Example 3

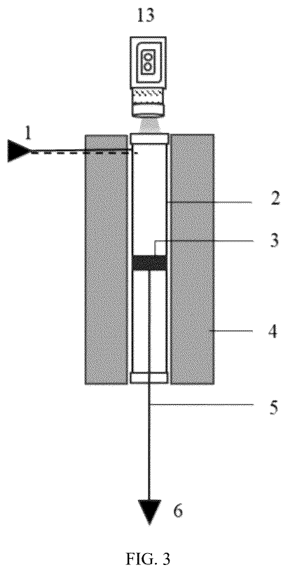

As shown in FIG. 3, it is an infrared temperature measurement system. The infrared temperature measurement system includes infrared thermal imaging 13. Infrared temperature measurement is based on the infrared radiation emitted by an object, and calculates the surface temperature of the object through the relationship between radiation power and temperature. Infrared temperature measurement in a reactor requires ensuring the passage of infrared radiation. As mentioned earlier, when the top of the fixed bed reaction tube 2 is replaced with germanium glass, germanium glass can ensure the passage of infrared radiation, allowing for temperature measurement of the catalytic bed layer through infrared temperature measurement. When accurately measuring the temperature of the catalytic bed, infrared thermography 13 can be used for temperature measurement.

### Example 4

Pd/TiO₂ Catalyst, Thermally Luminescent Material CaF₂

Firstly, add 0.1 g CaF₂ to the fixed bed reactor. Next, select 365 nm wavelength ultraviolet light with a xenon lamp and irradiate it for 15 minutes at a distance of 15 cm from CaF₂ at the top of the reaction tube under the irradiation intensity of 0.51 W/m². Next, 115 ml/min of N₂ was introduced under one atmosphere pressure, and the material was heated from room temperature to 250° C. at a rate of 5° C./min. Thermoluminescence spectroscopy was performed using a light signal detection system to obtain the relationship between temperature and the ratio of thermoluminescence intensity. Then, 0.1 g of Pd/TiO₂ catalyst was physically mixed with 0.1 g of CaF₂, and 365 nm wavelength ultraviolet light was selected using a xenon lamp. The reaction tube was irradiated for 15 minutes at a distance of 15 cm from CaF₂ at the top. Then, 100 ml/min of 1% C₂H₂/C₂H₄ and 15 ml/min of 10% H₂/N₂ were introduced under atmospheric pressure for hydrogenation reaction. The material was heated at a rate of 5° C./min from room temperature to 150° C., and the light intensity was detected using a light signal detection system. The actual temperature of the catalytic reaction was calculated by inputting the thermoluminescence intensity ratio at this temperature into the standard thermoluminescence temperature measurement curve.

### Example 5

PdCu MMO Catalyst and Pr³⁺ Doped Y₃Al₂Ga₃O₁₂ Thermally Luminescent Material

Firstly, 0.5 g of Pr³⁺ doped Y₃Al₂Ga₃O₁₂ was added to a fixed bed reactor. Then, 365 nm wavelength ultraviolet light was selected using a xenon lamp and irradiated for 15 minutes at a distance of 15 cm from Pr³⁺ doped Y₃Al₂Ga₃O₁₂ at the top of the reaction tube under an irradiation intensity of 0.51 W/m². Then, 60 ml/min of N₂ was introduced under one atmosphere pressure, and the material was heated from room temperature to 250° C. at a rate of 5° C./min. Thermoluminescence spectroscopy was performed using a light signal detection system to obtain the relationship between temperature and the ratio of thermoluminescence intensity. Then, 0.5 g of PdCu MMO catalyst was physically mixed with 0.5 g of Pr³⁺ doped Y₃Al₂Ga₃O₁₂. Similarly, 365 nm wavelength ultraviolet light was selected using a xenon lamp and irradiated for 15 minutes at a distance of 15 cm from the Pr³⁺ doped Y₃Al₂Ga₃O₁₂ at the top of the reaction tube. Then, 50 ml/min of 1% C₂H₂/C₂H₄ and 10 ml/min of 10% H₂/N₂ were introduced under atmospheric pressure for hydrogenation reaction. The material was heated at a rate of 5° C./min from room temperature to 200° C., and the light intensity was detected using a light signal detection system. The actual temperature of the catalytic reaction was calculated by inputting the thermoluminescence intensity ratio at this temperature into the standard thermoluminescence temperature measurement curve.

What is claimed is:

1. A fixed bed reactor suitable for in-situ heat removal and in-situ temperature measurement in gas-phase exothermic reactions, comprising a heating jacket and a fixed bed reaction tube, wherein
the heating jacket is installed outside the fixed bed reaction tube, a catalyst bed is installed inside the fixed bed reaction tube, a catalyst and a thermally luminescent material are placed inside the catalyst bed layer, two ends of the fixed bed reaction tube are respectively equipped with inlet and outlet ports, with inlet at the side and outlet at the lower end, a thermocouple is installed inside the fixed bed reaction tube, with one end extending to the catalyst bed and the other end extending outside the fixed bed reaction tube, and
the fixed bed reactor further comprises at least one of a xenon lamp pretreatment system, an optical signal detection system, and an infrared temperature measurement system.

2. The fixed bed reactor according to claim 1, wherein
the temperature and optical signal are measured at the top of the fixed bed reaction tube.

3. The fixed bed reactor according to claim 1, wherein
the xenon lamp pretreatment system comprises a xenon lamp, when pretreatment of thermally luminescent materials is required, align the xenon lamp probe with the catalyst bed inside the reaction tube, after irradiating the stored electrons, the catalytic bed is then heated by introducing an inert gas or reaction gas atmosphere.

4. The fixed bed reactor according to claim 1, wherein
the optical signal detection system comprises a reflector, a condenser, a monochromator, and a detector, when the top of the fixed bed reaction tube is made of quartz, measure the light intensity, the optical signal collection system is located at the top of the reaction tube, with the reflector and condenser in the darkroom, the thermochromic material in the catalytic bed emits light due to the release of electrons after heating up, which is refracted 90° by the reflector and converged by the condenser into the monochromator, the measured wavelength of light is selected and the intensity of the optical signal is measured by a detector, the detector converts the optical signal into an electrical signal to achieve optical signal intensity detection.

5. The fixed bed reactor according to claim 1, wherein
when the top of the fixed bed reaction tube is made of germanium glass, the temperature of the catalytic bed layer is measured by infrared temperature measurement.

6. A method for producing a fixed bed reactor according to claim 1, wherein
the method is used for acetylene hydrogenation to produce ethylene, the catalysts are Pd based, Ni based, Cu based catalysts, and the luminescent materials are rare earth doped calcium fluoride, lithium fluoride, calcium sulfate, and lithium borate, physically mixing the catalyst with thermally luminescent materials or doping or depositing rare earth elements onto the catalyst, followed by pretreatment and measurement of optical signals or temperature.
